(19) United States
(12) Patent Application Publication
(10) Pub. No.: US 2012/0177617 A1
(43) Pub. Date: Jul. 12, 2012

(54) Composition for Providing Relief of Undesired Body Odours

(76) Inventor: Carel Cristiaan van Helsdingen, Amsterdam (NL)

(21) Appl. No.: 13/383,100

(22) PCT Filed: Jul. 2, 2010
(86) PCT No.: PCT/NL10/50421

§ 371 Date: Mar. 30, 2012

(30) Foreign Application Priority Data
Jul. 9, 2009 (NL) 2003171

(51) Int. Cl. A61K 8/97; A61Q 90/00

(52) U.S. Cl. 424/93.7

(57) ABSTRACT

The present invention relates to a composition suitable for providing relief of undesired body odours caused by consumption of food and/or drink, comprising chlorella, parsley and a mouth refreshing agent. In particular, the food and/or drink comprises a plant, or part thereof, which belongs to the Allium genus. More in particular, the food and/or drink comprises onion, shallot, leek, bunching onion, chive, preferably garlic, any part thereof, or any combination thereof. The composition according to the invention comprises an amount of chlorella between 2 and 30, preferably between 4 and 25, more preferably between 5 and 20 percent by weight of the total composition.

The present invention relates to a composition providing relief of undesired body odours caused by consumption of food and/or drink and to the composition in the form of a tablet. Furthermore, the invention relates to a method of producing the composition.

Members from the botanical taxon of the Alliums, such as garlic, onion, leek, chives and the like, are consumed for their unique and attractive organoleptic properties which they provide when added to a meal.

Furthermore, several members of the Alliums, in particular garlic and onion, are also consumed for reasons related to their health promoting effects as a result of their antioxidant properties.

However, consumption of alliums, garlic in particular, is associated with the occurrence of unwanted smell escaping from the oral cavity and/or through the pores of the skin. Consumption of already small amounts of garlic is usually sufficient for such malodours to become noticeable in an undesired and pronounced manner. This odour or malodour escaping from the human body is perceived by most people in the Western world as a highly undesired, rather repulsive smell.

One marketed product which would alleviate garlic breath is green tea. Consumption of green tea is not wide-spread among young people likely because of the unfavourable taste or incompatibility with their lifestyle. Also, brewing of tea is considered a tedious, time-consuming activity for which many people do not have sufficient time or liking.

Consequently, there is a demand for a product which is more compatible with the modern lifestyle of busy people, or a product which appeals to young people, adolescents in particular and suits their lifestyle.

Therefore, an object of the present invention is to provide a composition suitable for providing relief of undesired body odours caused by consumption of food and/or drink. Furthermore, the object of the invention is to provide the composition in the form of a tablet. Also, an object of the present invention is to provide a method of producing the composition in the form of a tablet.

The above object, amongst others, is provided by a composition as defined in the appended claims.

Specifically, the present invention relates to a composition comprising chlorella, parsley and a mouth refreshing agent. This composition is suitable for providing relief of undesired body odours caused by consumption of food and/or drink.

The invention is particularly effective after consumption of food and/or drink which comprises a plant, or material thereof, which belongs to the Allium genus.

In a more preferred embodiment, the composition according to the invention is effective in providing relief after consumption of food and/or drink which comprises onion, shallot, leek, bunching onion, chive, preferably garlic, any part or parts thereof, or any combination thereof.

Malodour after consumption of a plant, or rather parts thereof, which belongs to the Alliums as mentioned herein, occurs, at least in case of garlic, in at least two separate forms, i.e. an immediate or primary form and a delayed secondary form. Immediately after consumption of garlic, significant levels of malodour escape from the oral cavity. The delayed form of malodour occurs from the day after consumption of garlic. This delayed effect results from the digestion and uptake of specific compounds comprised by garlic, uptake thereof by tissues of the gastrointestinal tract, metabolization of certain compounds and subsequent excretion from the skin or exhaling of particular odorizing substances. Also odours escaping from the gut or stomach can contribute to the undesired garlic breath. This latter form is thought to be a result of particular compounds comprised by garlic which are converted by microbes, bacteria in particular.

The manner in which relief of undesired body odours is achieved is based on a combination of several surprisingly effective mechanisms provided through specific components of the composition. Firstly, the pronounced perception of malodours which occurs directly after consumption of plants from the Allium family as mentioned herein, garlic in particular, is instantly diminished upon consumption of the composition according to the invention. This effect is provided through the mouth refreshing agent and chlorella which mask the presence and/or prevent the occurrence of such malodours.

Secondly, the composition prevents or reduces the excretion or escape of malodours from the human body that are caused by consumption of Allium plants as meant herein. Certain components comprised by the composition, in particular chlorella or particular substances comprised by chlorella, prevent or reduce the gastrointestinal uptake of malodour-causing compounds comprised by such Allium plants. Compounds that are prevented from being taken up through the intestinal tract no longer contribute to the perceived pronounced malodour which occurs after consumption of Allium plants. This effect is prominent and noticeable in reducing occurrence of malodour comprised by sweat or by escape from the skin and/or malodour from the oral cavity. Prevention of the uptake of substances that contribute to the malodour from the oral cavity aids in diminishing escape of undesired body odours. Such prevention can be by neutralising sulphur comprising compounds which are neutralised and consequently not taken up or taken up to a lesser extent.

Also, the perceptibility of any undesired body odour that originates from the lungs and/or gut and escapes through the oral cavity is effectively reduced or masked by the present composition. In particular, delayed intake of the composition according to the present invention, meaning for example intake the day after the consumption of the Allium plant comprising food and/or drink when a person may still suffer from malodour, alleviates such malodour and provides relief from such malodour.

In order for the composition to be effective in providing relief of malodours such by preventing the occurrence thereof, it is preferred that it is consumed within a sufficiently small time-frame centred around consumption of food and/or drink comprising an Allium plant or part thereof as meant herein. Such a time-frame spans from two, preferably one hour prior to the consumption of food and/or drink comprising the Allium plant or part thereof as meant herein to about two or preferably within one hour after consumption thereof. Obviously, the composition can also be taken within a shorter time period before or after consumption of the Allium-comprising food and/or drink. The composition can also be taken during the consumption of food and/or drink comprising the Allium plants, or parts thereof, as meant herein. The composition can also be taken the day after consumption of the Allium comprising food and/or drink but obviously in this case the uptake of Allium-comprised compounds that cause malodour is not prevented. However, in this latter case, the composition can still provide relief of Allium-associated malodour.

Not wishing to be bound by theory, the combination of chlorella and a mouth refreshing agent according to the invention provides an effective and suitable manner to provide relief of undesired body odours by reducing and/or preventing the occurrence or perceptibility thereof.

Chlorella herein is any suitable chlorella species such as Chlorella minutissima, Chlorella pyrenoidosa, Chlorella regularis, Chlorella reisiglii, Chlorella sorokiniana, Chlorella symbiont, Chlorella trebouxioides, Chlorella vulgaris or any combination thereof. The chlorella is preferably dried to a powder, which is in essence free of water but can comprise between 4-7% moist.

The term “food and/or drink” herein comprises any solid food or more liquid food such as a soup or a shake, consumed at any time of day or night.

Herein the terms “malodour” and undesired (body) odour” or the plural forms thereof are used interchangeably. Such malodours often occur after consumption of (parts from) plants from the Allium genus as meant herein. Such plants comprise substances that cause malodour, and/or substances that, after their digestion or metabolism by the human body, cause malodour. Malodour comprises both immediate and delayed forms, unless specified otherwise.

An “Allium plant” herein is a plant from the Allium genus and in preferred uses of the invention comprises onion, shallot, leek, bunching onion, chive, preferably garlic or any combination thereof. Wherever throughout the description “Allium plant” is written in the context of consumption of such a plant, it is also meant to include consumption of any part of such plant, such as a bulb, clove, shoot, leaf or leafy part.

In a preferred embodiment of the invention, the composition comprises any amount of chlorella between 2 and 30, preferably between 4 and 25, more preferably between 5 and 20 percent by weight of the total composition. Despite such relatively modest to considerable amounts of chlorella, an effective relief of malodour, in particular after consumption of Allium comprising food and/or drink, is still provided by the composition. Other such products, although not in particular used to provide relief of malodour after Allium-comprising food and/or drink, comprise chlorella as a main component (expressed as percentage by weight) or in essence consist of pure chlorella. Relative to this, the unique composition according to the invention comprise relatively modest amounts of chlorella but are still able to provide suitable relief of malodour. The specific amounts of chlorella according to the invention provide a green colour to the composition and a grassy, sharp, perhaps even unattractive, pronounced smell. The organoleptic properties of chlorella are also described as seaweed-like, both in taste and smell. An advantage of the preferred amounts of chlorella according to the invention is the provision of a lasting relief of malodour from consumption of Allium-comprising food and/or drink. Another advantage of using chlorella according to the invention is that it can be purchased and used very cost-effectively.

In another preferred embodiment, the composition further comprises parsley, preferably parsley leaf powder. Such parsley is preferably steam-treated prior its use. The addition of parsley provides another source of compounds to prevent or reduce malodour caused by consumption of Allium-comprising food. One such compound may be chlorophyll which is present in parsley as types a and b chlorophyll, as opposed to chlorella which comprises type c. Likely, also other compounds contribute to the relief of malodour, such as parsley-specific secondary metabolites or esoterical oils. However, not being bound by theory, it has been found that preferred amounts of parsley according to the invention provide a longer-lasting relief of malodour from consumption of Allium-comprising food and/or drink. Especially in the combination with chlorella according to the invention is an effective relief of malodour achieved.

Parsley is present in the composition in any amount between 2 and 30, preferably between 4 and 25, more preferably between 5 and 20 percent by weight of the total composition. Also these amounts are relatively modest compared to the use of pure parsley or other products that have parsley as a main component (expressed as percentage by weight) and would be useful to provide relief from garlic-associated malodour. The specific amounts of parsley according to the invention contribute to the green colour and grassy, spicy, pronounced smell of the composition.

In another preferred embodiment of the invention a sweetening agent is provided in the composition. In a more preferred embodiment, the sweetening agent is the principal component in terms of percentage by weight of the total composition. Suitable sweetening agents can be selected from the group consisting of xylitol, mannitol, arabitol, erythritol, isomalt, lactitol, maltitol or preferably sorbitol. The use of sorbitol is preferred as it is a nutritive sweetener providing a low amount of dietary energy (2.6 kilocalories) per gram having a suitable sweetening capacity of 0.6 (compared to sucrose) providing a preferred improved taste in combination with chlorella and/or parsley.

In a preferred embodiment of the invention, a sweetening agent is provided in any amount between 35 and 90, preferably between 40 and 85, more preferably between 50 and 85 percent by weight of the total composition. The sweetening agent is present in an amount in which preferred organoleptic properties are provided to the composition according to the invention. The relatively modest and effective amounts of chlorella and/or parsley allow for the presence of a sweetening agent as the main component expressed as percentage by weight. In this preferred combination according to the invention, which comprises chlorella, parsley, at least one mouth refreshing agent and sweetener in preferred amounts, a composition is provided with rather pronounced olfactorial properties which may not be perceived as an invitation to consume the composition, i.e. the composition has a rather sharp smell which may be associated with fermented grass or seaweed which stems from chlorella and/or parsley. Despite this strong smell, the disagreeable olfactorial properties do not negative the gustatory properties which are pleasant and candy-like with a minty sensation. Despite the candy-like flavour and mouth refreshing agent, the composition according to the invention is not meant to be taken as a candy or mint but should regarded more as a tablet or pastille which can be taken when relief or prevention is sought for a problem related to bad breath or unpleasant body odours.

The composition according to the invention thus seeks a balance between the requirements of providing an effective relief of malodour from consumption of Allium comprising food and/or drink by the low to moderate amounts of chlorella and/or parsley on the other hand and providing a suitable gustational sensation on the other hand which serves as an invitation for consumption of the composition. On top of this, the unpleasant olfactorial properties caused by chlorella and/or parsley are countered by the sweetening agent and mouth refreshing agent(s) such that the gustatorial properties of the composition do not suffer from the effective amounts of chlorella and/or parsley while not having made concessions to the malodour-relieving properties of the composition.

Another aspect of the invention relates to the mouth refreshing agent which in preferred embodiments is menthol, peppermint or a combination thereof. In more preferred embodiments the amount of menthol is any amount between 0.5 and 8, preferably between 1 and 6, more preferably between 1.5 and 4 percent by weight of the total composition and/or the amount of peppermint is any amount between 0.5 and 8, preferably between 1 and 6, more preferably between 1.5 and 4 percent by weight of the total composition. In preferred compositions of the invention, such mouth refreshing agents provide a short-lived relief of malodour.

Another aspect of the invention relates to a lubricant. An advantage of using a lubricant is that it allows suitable production of tablets. A lubricant reduces the stickiness of the composition thereby reducing the likelihood that the composition or ingredients thereof will stick to any manufacturing equipment when the composition is processed into an edible form, such as when being pressed into a tablet.

In particular, the lubricant is selected from the group consisting of sodium stearate, calcium stearate, potassium stearate, more in particular magnesium stearate or any combination thereof. With stearate herein is also meant the stearic acid form. The lubricant may also comprise palmitic acid comprising compounds or salts thereof. These lubricants may also have other suitable properties such as the capacity to bind sweetening agents thereby providing a firm and lasting binding effect.

In preferred embodiments of this aspect, the composition comprises an amount of lubricant of any amount between 0.1 and 2.0 percent by weight of the total composition. Such low amounts of lubricant allow for a composition to be made which comprises higher levels of other ingredients.

Yet another aspect of the invention relates to a preservative which optionally is part of the composition. In a preferred embodiment, such a preservative is citric acid. However, other preservatives are possible, as long as the flavour thereof is suitable for use in the composition. In a more preferred embodiment, the amount of preservative is any amount between 0.1 and 1.5 percent by weight of the total composition.

In order to reduce the taste and smell derived from Chlorella, in one embodiment of the present invention, the composition further comprises chlorophyllin. Addition of chlorophyllin allows a reduction in the amount of Chlorella, while relieve of undesired bodily odours can still be achieved. Preferably, the amount of chlorophyllin is in any amount of between 0.2 and 2, preferably 0.5 and 1.5 more preferably between 0.7 and 1.2 percent by weight of the composition.

In another preferred embodiment of the invention, the composition according to the present invention comprises the following components: chlorella in any amount between 2 and 30, preferably between 4 and 25, more preferably between 5 and 20 percent by weight; parsley in any amount between 2 and 30, preferably between 4 and 25, more preferably between 5 and 20 percent by weight; sweetening agent is any amount between 35 and 90, preferably between 40 and 85, more preferably between 50 and 85 percent by weight; menthol in any amount between 0.5 and 8, preferably between 1 and 6, more preferably between 1.5 and 4 percent by weight; peppermint in any amount between 0.5 and 8, preferably between 1 and 6, more preferably between 1.5 and 4 percent by weight; lubricant in any amount between 0.1 and 2.0 percent by weight; and optionally a preservative in any amount between 0.1 and 1.5 percent by weight of the total composition. These compounds are preferably in the form of a powder.

In yet another embodiment, the composition further comprises a flavouring agent or aromatising agent in a suitable amount. Such an agent can be selected from the group consisting of anise, licorice, anethole, anisole, citrus fruit flavour, lemon balm powder, citral, lemon flavour, pineapple flavour, strawberry flavour, apple flavour, pear flavour, banana flavour, orange flavour, cherry flavour, raspberry flavour, cinnamon, caramel, coriander, limonene, star anise flavouring, vanilla, cardamom, eucalyptus, honey, beeswax or any combination thereof. Such flavouring agents may be added as powder, isolated substance and/or synthetic substance.

In yet another embodiment, the composition further comprises a colouring agent in a suitable amount. Such an agent is preferably selected from natural colouring substances.

Yet another aspect of the invention relates to the composition in the form of a tablet, preferably a pressed tablet. Such a tablet, in a more preferred embodiment, is in essence without coating.

In another embodiment, the composition is provided with a coating. When applied, such a coating is preferably comprised of beeswax but other types of coating are also possible. The composition could also be supplied as a capsule comprising the composition in the form of a powder.

An advantage of having the composition in the form of a tablet or capsule, is that a directly usable product is made available which provides instantly effective relief of malodour which occurs after consumption of Allium-comprising food and/or drink, in particular garlic-comprising food and/or drink.

In this day and age in which people have busy and demanding social lifestyles, can a tablet according to the invention be of particular use. Especially adolescents, for which the tablet according to the invention is particularly intended, can benefit from the anywhere-anytime effect to obtain instant relief of malodour which the tablet according to the invention provides. Also people who do not wish to spend time for preparing green tea or chew on parsley leaves to get relief of Allium-associated malodour, there is a solution that complies better with their lifestyles which allows them to obtain instant relief of malodour, in particular garlic-associated malodour, anywhere-anytime.

The invention furthermore relates to the method of preparing the composition suitable for providing relief of undesired body odours caused by consumption of food and/or drink wherein the food and/or drink comprises a plant, or part thereof, which belongs to the Allium genus, more in particular, onion, shallot, leek, bunching onion, chive, preferably garlic, any part thereof, or any combination thereof, wherein the method comprises of mixing any or all of the components according to the invention and forming a pressed tablet. The parsley may be steamed and dried prior to the mixing of the components.

The invention is further specified by the following examples, which are not intended to limit the invention in any way.

### EXAMPLES

Several specific compositions were made with the purpose of preparing a composition which has preferred organoleptic properties. Composition 1 is preferred as a consequence of the preferred relief of garlic-associated malodour is provides and its preferred gustatorial properties. Composition one was perceived as herb-like or spicy at first, after which a rather sharp flavour was perceived and finally as providing a refreshing aftertaste.

TABLE 1
Composition 1.

| Ingredients | mg per tablet | % (w/w) |
|---|---|---|
| Chlorella powder | 62.5 | 8.2 |
| Parsley leaf powder | 50 | 6.6 |
| Peppermint flavour | 20 | 2.6 |
| Menthol flavor | 20 | 2.6 |
| Sorbitol | 600 | 79.0 |
| Magnesium Stearate | 7.5 | 1.0 |
| Total | 760 | 100 |

TABLE 2
Composition 2.

| Ingredients | mg per tablet | % (w/w) |
|---|---|---|
| Chlorophyllin | 12.5 | 0.9 |
| Parlsey leaf powder | 50 | 3.6 |
| Peppermint flavour | 9.8 | 0.7 |
| Isomalt | 1294.1 | 92.8 |
| Calcium Stearate | 28 | 2.0 |
| Total | 1394 | 100 |

TABLE 3
Composition 3.

| Ingredients | mg per tablet | % (w/w) |
|---|---|---|
| Chlorella powder | 62.5 | 8.6 |
| Parlsey leaf powder | 50 | 6.9 |
| Peppermint flavour | 10 | 1.4 |
| Sorbitol | 600 | 82.2 |
| Magnesium Stearate | 7.5 | 1.0 |
| Totaal | 730 | 100 |

TABLE 4
Composition 4.

| Ingredients | mg per tablet | % (w/w) |
|---|---|---|
| Chlorella powder | 62.5 | 8.6 |
| Parlsey leaf powder | 50 | 6.8 |
| Peppermint flavour | 7 | 1.0 |
| Menthol flavor | 4 | 0.6 |
| Sorbitol | 600 | 82.1 |
| Magnesium Stearate | 7.5 | 1.0 |
| Totaal | 731 | 100 |

TABLE 5
Composition 5.

| Ingredients | mg per tablet | % (w/w) |
|---|---|---|
| Chlorella powder | 62.5 | 8.5 |
| Parlsey leaf powder | 50 | 6.8 |
| Peppermint flavour | 11 | 1.5 |
| Menthol flavour | 4 | 0.5 |
| Citric Acid | 5 | 0.7 |
| Sorbitol | 600 | 81.1 |
| Magnesium Stearate | 7.5 | 1.0 |
| Totaal | 740 | 100 |

TABLE 6
Composition 6.

| Ingredients | mg per tablet | % (w/w) |
|---|---|---|
| Chlorella powder | 62.5 | 8.0 |
| Parlsey leaf powder | 50 | 6.4 |
| Peppermint flavour | 10 | 1.3 |
| Menthol flavour | 10 | 1.3 |
| Sucralose | 37 | 4.8 |
| Sorbitol | 600 | 77.2 |
| Magnesium Stearate | 7.5 | 1.0 |
| Totaal | 777 | 100 |

TABLE 7
Composition 7.

| Ingredients | mg per tablet | % (w/w) |
|---|---|---|
| Chlorella powder | 62.5 | 8.3 |
| Parlsey leaf powder | 50 | 6.6 |
| Peppermint flavour | 10 | 1.3 |
| Menthol flavour | 10 | 1.3 |
| Sucralose | 18.5 | 2.4 |
| Sorbitol | 600 | 79.1 |
| Magnesium Stearate | 7.5 | 1.0 |
| Totaal | 758.5 | 100 |

1-23. (canceled)

24. Composition suitable for providing relieve of undesired body odours caused by consumption of food and/or drink, comprising chlorella, parsley and a mouth refreshing agent.

25. The composition according to claim 24, wherein the food and/or drink comprises a plant, or part thereof, which belongs to the Allium genus.

26. The composition according to claim 24, wherein the food and/or drink comprises onion, shallot, leek, bunching onion, chive, preferably garlic, any part thereof, or any combination thereof.

27. The composition according to claim 24, wherein the amount of chlorella is any amount between 2 and 30, preferably between 4 and 25, more preferably between 5 and 20 percent by weight of the composition.

28. The composition according to claim 24, wherein the parsley is parsley leaf powder.

29. The composition according to claim 24, wherein the amount of parsley is any amount between 2 and 30, preferably between 4 and 25, more preferably between 5 and 20 percent by weight of the composition.

30. The composition according to claim 24, further comprising a sweetening agent, wherein the sweetening agent preferably is the principal component in percentage by weight of the composition.

31. The composition according to claim 24, wherein the sweetening agent is selected from the group consisting of xylitol, mannitol, arabitol, erythritol, isomalt, lactitol, maltitol or preferably sorbitol.

32. The composition according to claim 24, wherein the amount of sweetening agent is any amount between 35 and 90, preferably between 40 and 85, more preferably between 50 and 85 percent by weight of the composition.

33. The composition according to claim 24, wherein the mouth refreshing agent is menthol or peppermint or a combination thereof.

34. The composition according to claim 24, wherein the amount of menthol is any amount between 0.5 and 8, preferably between 1 and 6, more preferably between 1.5 and 4 percent by weight of the composition.

35. The composition according to claim 24, wherein the amount of peppermint is any amount between 0.5 and 8, preferably between 1 and 6, more preferably between 1.5 and 4 percent by weight of the composition.

36. The composition according to claim 24, further comprising a stearate comprising lubricant, in particular selected from the group consisting of sodium stearate, calcium stearate, potassium stearate, more in particular magnesium stearate or any combination thereof.

37. The composition according to claim 24, wherein the amount of lubricant is any amount between0.1 and 2 percent by weight of the composition.

38. The composition according to claim 24, further comprising a preservative.

39. The composition according to claim 24, wherein the amount of preservative is any amount between 0.1 and 1.5 percent by weight of the composition.

40. The composition according to claim 24, further comprising chlorophyllin, preferably in any amount of between 0.2 and 2, preferably 0.5 and 1.5 more preferably between 0.7 and 1.2 percent by weight of the composition.

41. The composition according to claim 24, comprising chlorella in any amount between 2 and 30, preferably between 4 and 25, more preferably between 5 and 20 percent by weight; parsley in any amount between 2 and 30, preferably between 4 and 25, more preferably between 5 and 20 percent by weight; sweetening agent is any amount between 35 and 90, preferably between 40 and 85, more preferably between 50 and 85 percent by weight; menthol in any amount between 0.5 and 8, preferably between 1 and 6, more preferably between 1.5 and 4 percent by weight; peppermint in any amount between 0.5 and 8, preferably between 1 and 6, more preferably between 1.5 and 4 percent by weight; lubricant in any amount between 0.1 and 2 percent by weight; and optionally a preservative in any amount between 0.1 and 1.5 percent by weight of the composition.

42. The composition according to claim 24, wherein the composition is in the form of a tablet, preferably a pressed tablet.

43. The composition according to claim 24, wherein the tablet is without coating.

44. The method of producing a composition suitable for providing relieve of undesired body odours caused by consumption of food and/or drink, comprising of mixing the components of the composition according to claim 24 and forming a pressed tablet.

45. The method according to claim 44, wherein the parsley is steamed and dried prior to the mixing of the components.

46. The method according to claim 44, wherein the components are in the form of a powder.
